(19) United States
(12) Patent Application Publication
(10) Pub. No.: US 2022/0200974 A1
(43) Pub. Date: Jun. 23, 2022

(54) SYSTEM AND METHOD FOR MERGING LIVE MEDICAL DEVICE READINGS INTO VIRTUAL DOCTOR VISIT SECURE VIDEO

(71) Applicant: ARRIS Enterprises LLC, Suwanee, GA (US)

(72) Inventor: William W. JENNINGS, Brookhaven, GA (US)

(21) Appl. No.: 17/471,925

(22) Filed: Sep. 10, 2021

Related U.S. Application Data
(60) Provisional application No. 63/161,085, filed on Mar. 15, 2021.
(60) Provisional application No. 63/126,742, filed on Dec. 17, 2020.

(51) Int. Cl. H04L 29/06; H04N 7/15; G16H 80/00; G16H 40/67

(52) U.S. Cl. CPC H04L 63/0457; G16H 40/67; G16H 80/00; H04N 7/15

(57) ABSTRACT

A system and method are provided for a detector device for use with a first video conference device, a second video conference device, and WAN. The video conference devices are configured to: establish a video conference over a secure communication channel over the WAN; to encode user video/audio data; to encrypt the encoded data; to provide the encrypted data to the other video conference device; to receive encrypted data; to decrypt the encrypted data; decode the encoded data; instruct the display to display video data based on the decoded video data; and instruct the speaker to play audio data based on the decoded audio data. The first video conference device is additionally configured to: receive detector data from the detector device; to encode detector data; to encrypt the encoded data; and to provide the encrypted data to the other video conference device.

### BACKGROUND

Embodiments of the invention relate to video conferencing.

### SUMMARY

Aspects of the present disclosure are drawn to a first video conference device for use with a detector device, a second video conference device, and a wide area network (WAN), the second video conference device being configured to perform a video conference over a secure communication channel over the WAN, to encode second user video data and second user audio data to obtain second encoded data, to encrypt the second encoded data to obtain second encrypted data, to provide the second encrypted data to the first video conference device during the video conference, to receive first encrypted data, to display first user video data based on the first encrypted data, to provide detector data and to play first user audio data based on the first encrypted data, the detector device being configured to provide the detector data to the first video conference device, the first video conference device including: a camera configured to generate the first user video data; a microphone configured to generate the first user audio data; a display; a speaker; a memory; and a processor configured to execute instructions stored on the memory to cause the first video conference device to: receive the first user video data from the camera; receive the first user audio data from the microphone; receive the detector data from the detector device; encode the first user video data, the first user audio data and the detector data to generate the first encoded data; encrypt the first encoded data to generate first encrypted data; transmit the first encrypted data to the second video conference device during the video conference; receive the second encrypted data; decrypt the second encrypted data to obtain second encoded data; decode the second encoded data to obtain the second video data and the second audio data; instruct the display to display second video data based on the second video data; and instruct the speaker to play second audio data based on the second audio data.

In some embodiments, the processor is configured to execute instructions stored on the memory to additionally cause the first video conference device to encode the first user video data, the first user audio data and the detector data to generate the first encoded data such that the detector data is encapsulated within at least one of the first user video data and the first user audio data.

In some embodiments, the processor is configured to execute instructions stored on the memory to additionally cause the first video conference device to: encode the first user video data and the first user audio data to generate the encoded first user video data and audio data; encode the detector data to generate encoded detector data; encrypt the first user video data and the first user audio data to generate encrypted first user data; encrypt the encoded detector data to generate encrypted detector data; transmit the encrypted first user data to the second video conference device via a first communication protocol; and transmit the encrypted detector data to the second video conference device via a second communication protocol.

In some embodiments, the first video conference device is configured for use with the detector device being configured to detect a biological parameter and provide a detected signal based on the detected biological parameter. In some of these embodiments, the first video conference device is configured for use with the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

Other aspects of the present disclosure are drawn to a method of using a first video conference device with a detector device, a second video conference device, and a WAN, the second video conference device being configured to perform a video conference over a secure communication channel over the WAN, to encode second user video data and second user audio data to obtain second encoded data, to encrypt the second encoded data to obtain second encrypted data, to provide the second encrypted data to the first video conference device during the video conference, to receive first encrypted data, to display first user video data based on the first encrypted data, to provide detector data and to play first user audio data based on the first encrypted data, the detector device being configured to provide the detector data to the first video conference device, the method including: receiving, from a camera and via a processor configured to execute instructions stored on a memory, first user video data; receiving, from a microphone and via the processor, first user audio data; receiving, from the detector device and via the processor, the detector data; encoding, via the processor, the first user video data, the first user audio data and the detector data to generate the first encoded data; encrypting, via the processor, the first encoded data to generate first encrypted data; transmitting, via the processor, the first encrypted data to the second video conference device during the video conference; receiving, via the processor, the second encrypted data; decrypting, via the processor, the second encrypted data to obtain second encoded data; decoding, via the processor, the second encoded data to obtain the second video data and the second audio data; instructing, via the processor, a display to display second video data based on the second video data; and instructing, via the processor, a speaker to play second audio data based on the second audio data.

In some embodiments, the present disclosure is drawn to encoding, via the processor, the first user video data, the first user audio data and the detector data to generate the first encoded data includes encoding the first user video data, the first user audio data and the detector data to generate the first encoded data such that the detector data is encapsulated within at least one of the first user video data and the first user audio data.

In some embodiments, the method further includes: encrypting, via the processor, the first user video data and the first user audio data to generate encrypted user data; encoding, via the processor, the first user video data and the first user audio data to generate encoded first user video data and encoded first user audio data; encoding, via the processor, the detector data to generate encoded detector data; encrypting, via the processor, the first user video data and the first user audio data to generate encrypted first user data; encrypting, via the processor, the encoded detector data to generate encrypted detector data; transmitting, via the processor, the encrypted first user data to the second video conference device via a first communication protocol; and transmitting, via the processor, the encrypted detector data to the second video conference device via a second communication protocol.

In some embodiments the receiving the detector data includes receiving detector data from the detector device being configured to detect a biological parameter and provide a detected signal based on the detected biological parameter. In some of these embodiments, the receiving the detector data includes receiving detector data from the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

Other aspects of the present disclosure are drawn to a non-transitory, computer-readable media having computer-readable instructions stored thereon, the computer-readable instructions being capable of being read by a first video conference device for use with a detector device, a second video conference device, and a WAN, the second video conference device being configured to perform a video conference over a secure communication channel over the WAN, to encode second user video data and second user audio data to obtain second encoded data, to encrypt the second encoded data to obtain second encrypted data, to provide the second encrypted data to the first video conference device during the video conference, to receive first encrypted data, to display first user video data based on the first encrypted data, to provide detector data and to play first user audio data based on the first encrypted data, the detector device being configured to provide the detector data to the first video conference device, wherein the computer-readable instructions are capable of instructing the first video conference device to perform the method including: receiving, from a camera and via a processor configured to execute instructions stored on a memory, first user video data; receiving, from a microphone and via the processor, first user audio data; receiving, from the detector device and via the processor, the detector data; encoding, via the processor, the first user video data, the first user audio data and the detector data to generate the first encoded data; encrypting, via the processor, the first encoded data to generate first encrypted data; transmitting, via the processor, the first encrypted data to the second video conference device during the video conference; receiving, via the processor, the second encrypted data; decrypting, via the processor, the second encrypted data to obtain second encoded data; decoding, via the processor, the second encoded data to obtain the second video data and the second audio data; instructing, via the processor, a display to display second video data based on the second video data; and instructing, via the processor, a speaker to play second audio data based on the second audio data.

In some embodiments, the computer-readable instructions are capable of instructing the first video conference device to perform the method wherein the encoding, via the processor, the first user video data, the first user audio data and the detector data to generate the first encoded data includes encoding the first user video data, the first user audio data and the detector data to generate the first encoded data such that the detector data is encapsulated within at least one of the first user video data and the first user audio data.

In some embodiments, the computer-readable instructions are capable of instructing the first video conference device to perform the method further including: encrypting, via the processor, the first user video data and the first user audio data to generate encrypted user data; encrypting, via the processor, the detector data to generate encrypted detector data; encoding, via the processor, the encrypted user data to generate the encrypted encoded user video data and encrypted encoded user audio data; encoding, via the processor, the encrypted detector data to generate the encrypted encoded detector data; transmitting, via the processor, the encrypted encoded user video data and encrypted encoded user audio data to the second video conference device via a first communication protocol; and transmitting, via the processor, the encrypted encoded detector data to the second video conference device via a second communication protocol.

In some embodiments, the computer-readable instructions are capable of instructing the first video conference device to perform the method wherein the receiving the detector data includes receiving detector data from the detector device being configured to detect a biological parameter and provide a detected signal based on the detected biological parameter. In some of these embodiments, the computer-readable instructions are capable of instructing the first video conference device to perform the method wherein the receiving the detector data includes receiving detector data from the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

### BRIEF SUMMARY OF THE DRAWINGS

The accompanying drawings, which are incorporated in and form a part of the specification, illustrate example embodiments and, together with the description, serve to explain the principles of the present disclosure. In the drawings:

FIG. 1 illustrates a video conference system in accordance with aspects of the present disclosure;

FIG. 2 illustrates an algorithm that merges live medical device readings into a secure video conference in accordance with aspects of the present disclosure;

FIG. 3 illustrates an exploded view of a service provider server, a gateway device, a video conference device, and a detector device in accordance with aspects of the present disclosure;

FIG. 4A illustrates an exploded view of a processor at a time t₀ in accordance with aspects of the present disclosure;

FIG. 4B illustrates an exploded view of a processor at a time t₁ in one sample embodiment in accordance with aspects of the present disclosure;

FIG. 4C illustrates an exploded view of a processor at a time t₂ in one sample embodiment in accordance with aspects of the present disclosure;

FIG. 5 illustrates an exploded view of a service provider server, a gateway device and a video conference device in accordance with aspects of the present disclosure;

FIG. 6A illustrates an exploded view of a processor at a time t₀ in accordance with aspects of the present disclosure;

FIG. 6B illustrates an exploded view of a processor at a time t₁ in one sample embodiment in accordance with aspects of the present disclosure;

FIG. 6C illustrates an exploded view of a processor at a time t₂ in one sample embodiment in accordance with aspects of the present disclosure;

FIGS. 7A and 7B illustrate sample embodiments of a secure video conference in accordance with aspects of the present disclosure;

FIG. 7C illustrates a sample embodiment of a secure video conference in accordance with aspects of the present disclosure;

FIG. 8 illustrates a packet stream in accordance with aspects of the present disclosure;

FIG. 9 illustrates an exploded view of packet 802 in accordance with aspects of the present disclosure; and

FIG. 10 illustrates an exploded view of packet header 902 in accordance with aspects of the present disclosure.

### DETAILED DESCRIPTION

Due to a global pandemic, it has become increasingly difficult to have in-person doctor appointments. However, the increase in technology has allowed for many people to get the same service through a secure teleconference. Patients in one location can use a video conferencing device to interact with their doctor in another location using their own device. However, a problem arises when the doctor needs to check vital biometric data of the patient using equipment, and it may be hard for the patient to show the doctor their biometric data.

What is needed is a system and method for combining detector data with an existing secure teleconference to make it easy for a doctor to check a patient's biometric data during a virtual doctor's visit.

A system and method in accordance with the present disclosure combines detector data with an existing secure teleconference.

A proposed solution to the problem described above will allow a patient to transmit their biometric data to a doctor during a teleconference. During the teleconference, the patient will attach medical equipment to themselves, such as blood pressure cuffs, thermometers, etc. Then, via wired or wireless methods, the biometric data from the medical equipment will be transmitted to the streaming device of the patient. Finally, the biometric data is directly embedded into the video stream to the doctor. This will ensure that the biometric data is correctly and safely given to the doctor.

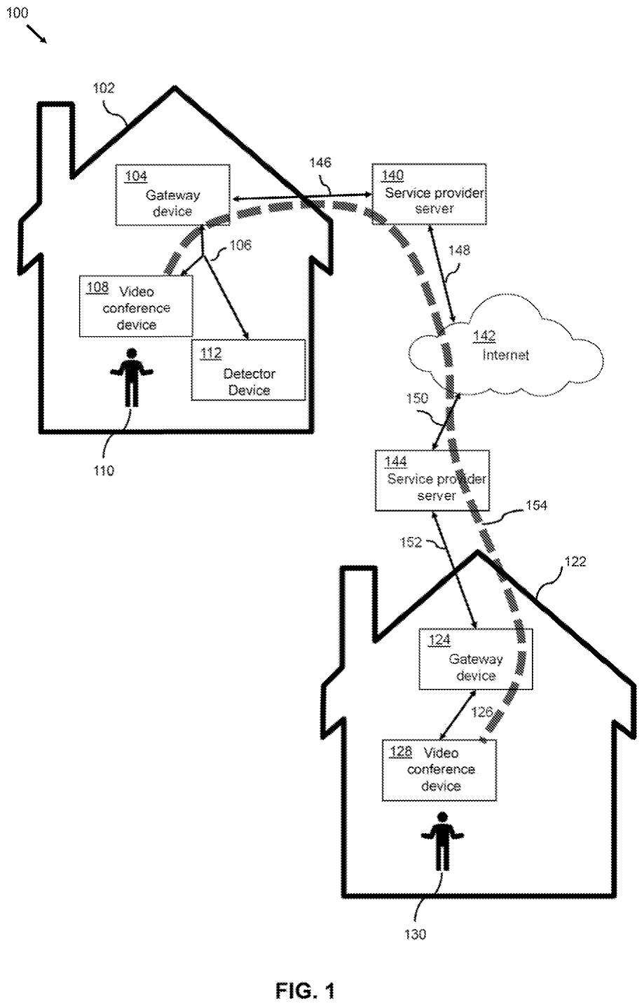

FIG. 1 illustrates a video conference system 100 in accordance with aspects of the present disclosure.

As shown in the figure, system 100 includes gateway devices 104 and 124, video conference devices 108 and 128, users 110 and 130, and a detector device 112. Gateway device 104, video conference device 108, user 110, and detector device 112 are positioned in location 102; gateway device 124, video conference device 128, and user 130 are positioned in location 122. A communications channel 106 connects gateway device 104, video conference device 108, and detector device 112. A communications channel 126 connects gateway device 124 and video conference device 128. Gateway device 104 connects to an Internet 142 through a service provider server 140 using communications channels 146 and 148, while gateway device 124 connects to Internet 142 through a service provider server 144 using communications channels 150 and 152. A secure communications channel 154 is established between video conference devices 108 and 128, using gateway devices 104 and 124, service providers 140 and 144, and Internet 142 over communications channels 106, 146, 148, 150, 152, and 126.

Gateway devices 104 and 124, also referred to as gateways, residential gateways, or RGs, are electronic devices that are located so as to establish local area networks (LANs) at locations 102 and 122. Locations 102 and 122 can include residential dwellings, offices, or any other business space of users 110 and 130. The terms home, office, and premises may be used synonymously herein.

Gateway devices 104 and 124 may be any devices or systems that are operable to allow data to flow from one discrete device or network to another. Gateway devices 104 and 124 may perform such functions as Web acceleration and HTTP compression, flow control, encryption, redundancy switchovers, traffic restriction policy enforcement, data compression, TCP performance enhancements (e.g., TCP spoofing), quality of service functions (e.g., classification, prioritization, differentiation, random early detection, TCP/UDP flow control), bandwidth usage policing, dynamic load balancing, address translation, and routing. In this non-limiting example, gateway devices 104 and 124 may be routers, gateways, extenders, or mesh network devices.

Video conference devices 108 and 128 are any devices or systems that are able to establish a video conference wherein video and audio data from video conference device 108 is presented on video conference device 128 and video and audio data from video conference device 128 is presented on video conference device 108. In this non-limiting example, video conference devices 108 and 128 may be smart phones, tablets, personal computers, or smart media devices.

Detector device 112 may be any known detector device that is configured to detect a biological parameter and provide a detected signal based on the detected biological parameter. Non-limiting examples of detector devices include cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

Service provider servers 140 and 144 include head-end equipment such as server computers (e.g., automatic configuration server ACS, cable modem termination system CMTS) that enable service provider servers 140 and 144, such as cable television providers, satellite television providers, internet service providers, or multiple-systems operators (MSOs), to provide content such as audio/video content and/or internet service through communication channels 146 and 152 utilizing physical media/wiring such as coaxial networks, optical fiber networks, or DSL; or wireless infrastructure such as satellites, terrestrial antennas, or any combination of these examples or their equivalents.

Communication channels 106, 146, 148, 150, 152, and 126 are any devices or systems that facilitate communications between devices or networks. In this non-limiting example, communication channels 106 and 126 are Wi-Fi or Bluetooth channels. The term “Wi-Fi” as used herein may be considered to refer to any of Wi-Fi 4, 5, 6, 6E, or any variation thereof. The term “Bluetooth” as used herein may be considered to refer to Classic Bluetooth, Bluetooth high speed, or Bluetooth Low Energy (BLE) protocols, or any variation thereof. Communication channels 106, 146, 148, 150, 152, and 126 may include physical media or wiring, such as coaxial cable, optical fiber, or digital subscriber line (DSL); or wireless links, such as LTE, satellite, or terrestrial radio links; or a combination of any of these examples or their equivalents. The data communicated on such networks can be implemented using a variety of protocols on a network such as a WAN, a virtual private network (VPN), a metropolitan area network (MAN), a system area network (SAN), a DOCSIS network, a fiber optics network (including fiber-to-the-home, fiber-to-the-X, or hybrid fiber-coax), a digital subscriber line (DSL), a public switched data network (PSDN), a global Telex network, or a 2G, 3G, 4G or 5G network, for example. Though communication channels 106, 146, 148, 150, 152, and 126 are shown as single links, it is contemplated that communication channels 106, 146, 148, 150, 152, and 126 may contain multiple links and devices including access points, routers, gateways, and servers.

User 110 is a person using video conference device 108 at location 102. In this non-limiting example, user 110 is a patient. User 130 is a person using video conference device 128 at location 122. In this non-limiting example, user 130 is a doctor.

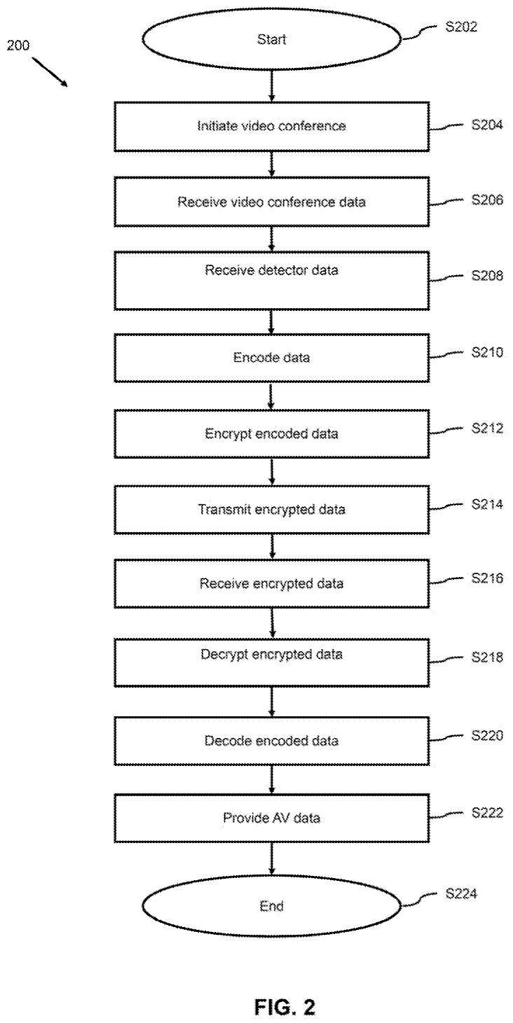

In normal operation, video conference device 108 establishes secure communication channel 154 to video conference device 128. User 130 conducts a virtual doctor's visit with user 110 using video conference devices 108 and 128 over secure communication channel 154. This will be described in greater detail with reference to FIG. 2.

FIG. 2 illustrates algorithm 200 to be executed on a processor that merges live medical device readings into a secure video conference in accordance with aspects of the present disclosure.

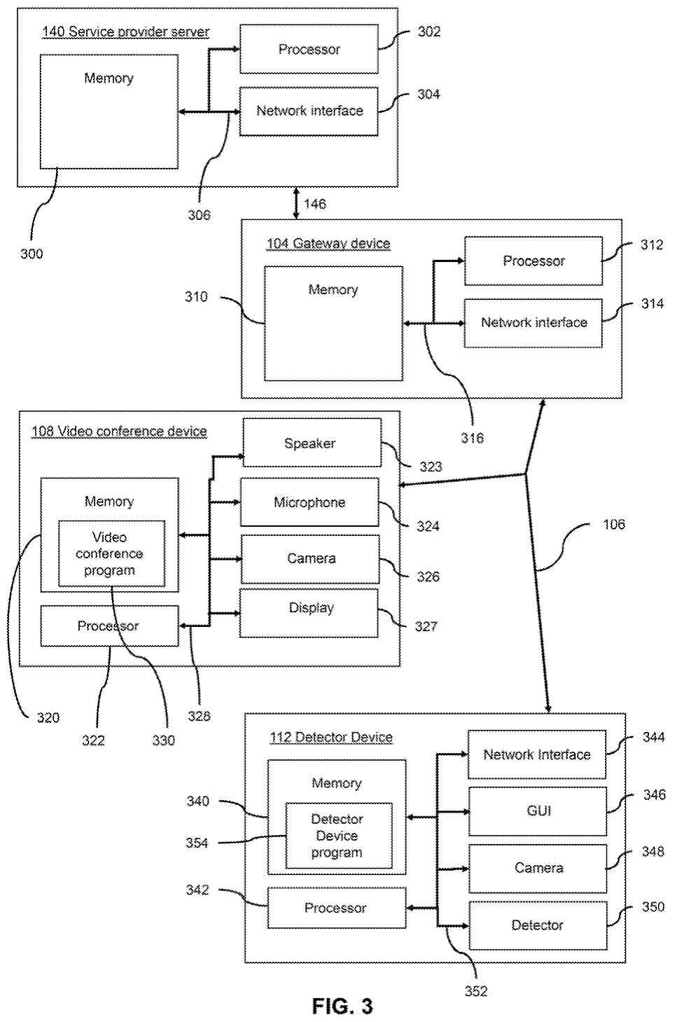

As shown in the figure, algorithm 200 starts (S202), and a video conference is initiated (S204). This will be described in greater detail with reference to FIG. 3.

FIG. 3 illustrates an exploded view of service provider server 140, gateway device 104, video conference device 108, and detector device 112 in accordance with aspects of the present disclosure.

As shown in the figure, service provider server 140 contains a memory 300, a processor 302, and a network interface 304. Memory 300, processor 302, and network interface 304 are connected by a bus 306. Gateway device 104 contains a memory 310, a processor 312, and a network interface 314. Memory 310, processor 312, and network interface 314 are connected by a bus 316. Video conference device 108 contains a memory 320, a processor 322, a speaker 323, a microphone 324, a camera 326, and a display 327. Memory 320, processor 322, speaker 323, microphone 324, camera 326, and display 327 are connected by bus 328. A video conference program 330 is contained in memory 320 and is executed by processor 322. Detector device 112 contains a memory 340, a processor 342, a network interface 344, a GUI 346, a camera 348, and a detector 350. Memory 340, processor 342, network interface 344, GUI 346, camera 348, and detector 350 are connected by bus 352. A detector device program 354 is contained in memory 340 and is executed by processor 342.

Though only service provider server 140, gateway device 104, and video conference device 108 are shown in FIG. 3, service provider server 144 is substantially similar to service provider server 140, gateway device 124 is substantially similar to gateway device 104, and video conference device 128 is substantially similar to video conference device 108.

Processors 302, 312, 322, and 342 are any devices or systems capable of controlling general operations of devices 140, 104, 108, and 112 respectively, and include, but are not limited to, central processing units (CPUs), hardware microprocessors, single-core processors, multi-core processors, field-programmable gate arrays (FPGAs), microcontrollers, application-specific integrated circuits (ASICs), digital signal processors (DSPs), or other similar processing devices capable of executing any type of instructions, algorithms, or software for controlling the operation and functions of devices 140, 104, 108, and 112.

Memories 300, 310, 320, and 340 are any devices or systems capable of storing data and instructions used by devices 140, 104, 108, and 112 respectively, and include, but are not limited to, random-access memory (RAM), dynamic random-access memory (DRAM), hard drives, solid-state drives, read-only memory (ROM), erasable programmable read-only memory (EPROM), electrically erasable programmable read-only memory (EEPROM), flash memory, embedded memory blocks in FPGAs, or any other various layers of memory hierarchy.

Network interfaces 304, 314, and 344 are any devices or systems used to establish and maintain communication channels 146 and 106. Network interfaces 304, 314, and 344 may include one or more antennas and communicate wirelessly via one or more of the 2.4 GHz band, the 5 GHz band, the 6 GHz band, and the 60 GHz band, or at the appropriate band and bandwidth to implement any IEEE 802.11 Wi-Fi protocols, such as the Wi-Fi 4, 5, 6, or 6E protocols. Devices 104 and 112 can also be equipped with radio transceivers or wireless communication circuits to implement wireless connections in accordance with any Bluetooth protocols, Bluetooth Low Energy (BLE), or other short-range protocols that operate in accordance with a wireless technology standard for exchanging data over short distances using any licensed or unlicensed band such as the CBRS band, 2.4 GHz bands, 5 GHz bands, 6 GHz bands, or 60 GHz bands, RF4CE protocol, ZigBee protocol, Z-Wave protocol, or IEEE 802.15.4 protocol.

In this non-limiting example, detector device 112 is configured to communicate with video conference device 108 via communication channel 106. However, in other example embodiments, detector device 112 may communicate with video conference device 108 via a direct wired connection.

GUI 346 is any device or system capable of presenting information and accepting user inputs on detector device 112, and includes, but is not limited to, liquid crystal displays (LCDs), thin film transistor (TFT) displays, light-emitting diode (LED) displays, or other similar display devices, including display devices having touch screen capabilities so as to allow interaction between user 110 and detector device 112.

Speaker 323 is any device or system that emits sound.

Microphone 324 is any device that converts sound into data to be transmitted.

Cameras 326 and 348 are any devices or systems that form an image.

Display 327 may be any known device or system to display an image to the user.

Detector 350 may be any known detector that is configured to detect a biological parameter and provide a detected signal based on the detected biological parameter. Non-limiting examples of detectors include cameras, microphones, pressure sensors, blood-pressure sensors, chemical detectors, oxygen sensors, carbon dioxide sensors, heart sound sensors, blood-flow sensors, respiration sensors, electrochemical electrodes, electrocardiograms, and combinations thereof.

In this example, processor 342, memory 340, network interface 344, GUI 346, camera 348, and detector 350 are illustrated as individual components of detector device 112. However, in some embodiments, at least two of processor 342, memory 340, network interface 344, GUI 346, camera 348, and detector 350 may be combined as a unitary device. Whether as individual devices or as combined devices, processor 342, memory 340, network interface 344, GUI 346, camera 348, and detector 350 may be implemented as any combination of an apparatus, a system and an integrated circuit. Further, in some embodiments, at least one of processor 342, memory 340, and network interface 344 may be implemented as a computer having non-transitory computer-readable media for carrying or having computer-executable instructions or data structures stored thereon. Such non-transitory computer-readable recording medium refers to any computer program product, apparatus or device, such as a magnetic disk, optical disk, solid-state storage device, memory, programmable logic devices (PLDs), DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media. For information transferred or provided over a network or another communications connection (either hardwired, wireless, or a combination of hardwired or wireless) to a computer, the computer may properly view the connection as a computer-readable medium. Thus, any such connection may be properly termed a computer-readable medium. Combinations of the above should also be included within the scope of computer-readable media.

Example tangible computer-readable media may be coupled to processor 342 such that the processor may read information from, and write information to the tangible computer-readable media. In the alternative, the tangible computer-readable media may be integral to processor 342. Processor 342 and the tangible computer-readable media may reside in an integrated circuit (IC), an ASIC, or large scale integrated circuit (LSI), system LSI, super LSI, or ultra LSI components that perform a part or all of the functions described herein. In the alternative, processor 342 and the tangible computer-readable media may reside as discrete components.

Example tangible computer-readable media may be also coupled to systems, non-limiting examples of which include a computer system/server, which is operational with numerous other general purpose or special purpose computing system environments or configurations. Examples of well-known computing systems, environments, and/or configurations that may be suitable for use with computer system/server include, but are not limited to, personal computer systems, server computer systems, thin clients, thick clients, handheld or laptop devices, multiprocessor systems, microprocessor-based systems, set-top boxes, programmable consumer electronics, network PCs, minicomputer systems, mainframe computer systems, and distributed cloud computing environments that include any of the above systems or devices, and the like.

Such a computer system/server may be described in the general context of computer system-executable instructions, such as program modules, being executed by a computer system. Generally, program modules may include routines, programs, objects, components, logic, data structures, and so on that perform particular tasks or implement particular abstract data types. Further, such a computer system/server may be practiced in distributed cloud computing environments where tasks are performed by remote processing devices that are linked through a communications network. In a distributed cloud computing environment, program modules may be located in both local and remote computer system storage media including memory storage devices.

Similar structures and combinations may exist for components of service provider server 144, gateway device 124, and video conference device 128, with reference to FIG. 1.

Bus 352 is any device or system that provides data communications between processor 342, memory 340, network interface 344, GUI 346, camera 348, and detector 350 of detector device 112. Bus 352 can be one or more of any of several types of bus structures, including a memory bus or a memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus. Similar relationships define buses 306, 316, and 328 contained in service provider server 140, gateway device 104, and video conference device 108, respectively, with reference to FIG. 1.

Detector device program 354 establishes and maintains the video conference session on detector device 112. Detector device program 354, having a set (at least one) of program modules, may be stored in memory 340 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

Video conference program 330 establishes and maintains the video conference session on video conference device 108. Video conference program 330, having a set (at least one) of program modules, may be stored in memory 320.

In some embodiments, as will be described in greater detail below, processor 322 is configured to execute instructions stored in memory 320 to cause video conference device 108 to receive the first user video data from camera 326; receive the first user audio data from microphone 324; receive the detector data from detector 350; encode the first user video data, the first user audio data and the detector data to generate the first encoded data; encrypt the first encoded data to generate first encrypted data; transmit the first encrypted data to video conference device 128 during the video conference; receive the second encrypted data; decrypt the second encrypted data to obtain second encoded data; decode the second encoded data to obtain the second video data and the second audio data; instruct display 327 to display second video data; and instruct speaker 323 to play second audio data.

In some embodiments, as will be described in greater detail below, processor 322 is additionally configured to execute instructions stored in memory 320 to additionally cause video conference device 108 to encode the first user video data, the first user audio data and the detector data to generate the first encoded data such that the detector data is encapsulated within at least one of the first user video data and the first user audio data.

In some embodiments, as will be described in greater detail below, processor 322 is additionally configured to execute instructions stored in memory 320 to additionally cause video conference device 108 to encode the first user video data and the first user audio data to generate encoded first user video data and encoded first user audio data; encode the detector data to generate encoded detector data; encrypt the first user video data and the first user audio data to generate encrypted first user data; encrypt the encoded detector data to generate encrypted detector data; transmit the encrypted first user data to video conference device 128 via a first communication protocol; and transmit the encrypted detector data to video conference device 128 via a second communication protocol.

In some embodiments, as will be described in greater detail below, processor 322 is additionally configured to execute instructions stored in memory 320 to additionally cause video conference device 108 to be used with detector device 112 being configured to detect a biological parameter and provide a detected signal based on the detected biological parameter. In some of these embodiments, as will be described in greater detail below, processor 322 is additionally configured to execute instructions stored in memory 320 to additionally cause video conference device 108 to be used with detector device 112 being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

With reference to FIGS. 1 and 2, presume that user 110 would like to initiate a virtual doctor visit with user 130. After secure communication channel 154 is established, user 110 will use video conference device 108, where processor 322 will execute video conference program 330 of memory 320. User 130 will perform a similar process using video conference device 128, and the virtual doctor visit will be initiated.

It should be noted that in some embodiments, user 130 can initiate the virtual doctor visit.

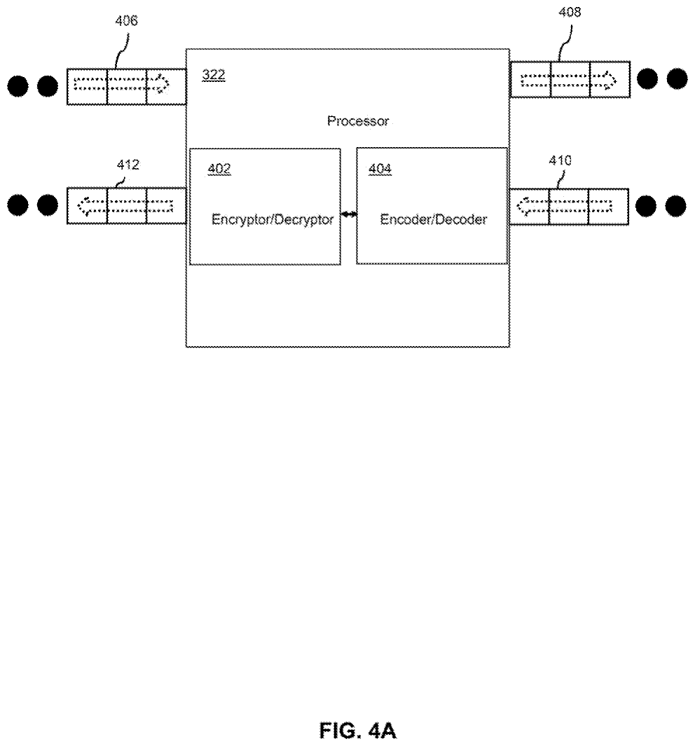

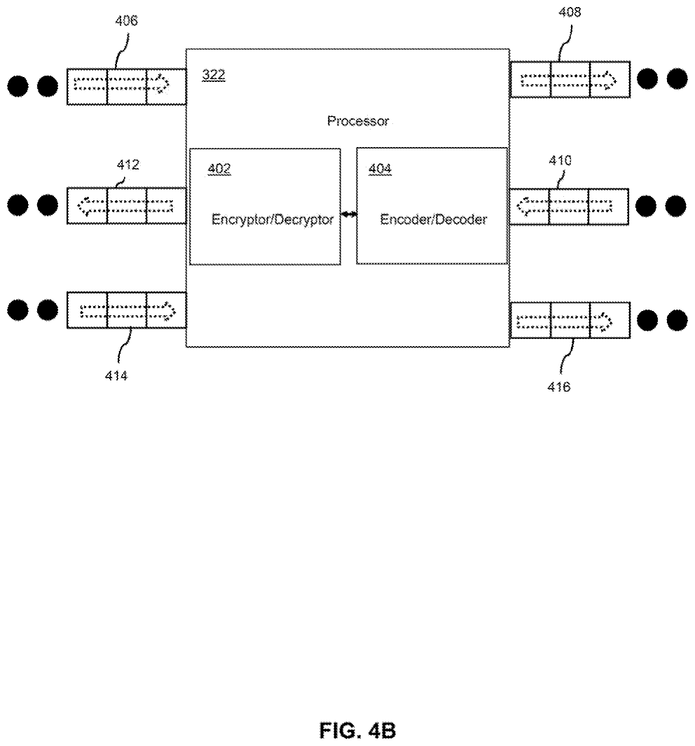

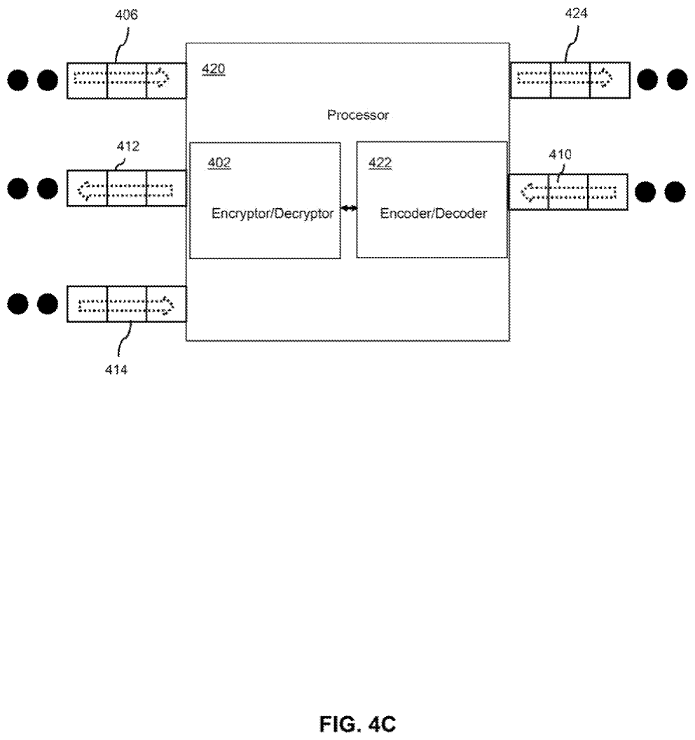

Returning to FIG. 2, after a video conference is initiated (S204), video conference data is received (S206). This will be described in greater detail with reference to FIG. 4A.

FIG. 4A illustrates an exploded view of processor 322 at a time t₀ in accordance with aspects of the present disclosure.

As shown in the figure, processor 322 includes an encryptor/decryptor 402, an encoder/decoder 404, and packet streams 406, 408, 410, and 412. Encryptor/decryptor 402 is arranged to communicate with encoder/decoder 404.

In this example, encryptor/decryptor 402 and encoder/decoder 404 are illustrated as individual components of processor 322. However, in some embodiments, encryptor/decryptor 402 and encoder/decoder 404 may be combined as a unitary device. Further, in some embodiments, at least one of encryptor/decryptor 402 and encoder/decoder 404 may be implemented as a computer having non-transitory computer-readable media for carrying or having computer-executable instructions or data structures stored thereon.

Encryptor/decryptor 402 encrypts sensitive data being transmitted from processor 322, which will be ultimately transmitted to video conference device 128, and decrypts encrypted data that is transmitted to processor 322, which will be received from video conference device 128.

Encoder/decoder 404 will convert encrypted data into binary code to be transmitted out of processor 322, which will be ultimately transmitted to video conference device 128. Further, encoder/decoder 404 will decode encoded data that is transmitted to processor 322, which will be received from video conference device 128.

For example, presume that the virtual doctor visit between user 110 and user 130 has been initiated. Video conference device 108 will receive video and audio data from user 110, and video conference device 128 will receive video and audio data from user 130. As shown in FIG. 4A, the video data from camera 326 and the audio data from microphone 324 will be transmitted to processor 322, shown as packet stream 406. Video conference device 128 will act in a similar manner.

Returning to FIG. 2, after video conference data is received (S206), detector data is received (S208). This will be described in greater detail with reference to FIGS. 1, 3 and 4B.

As shown in FIG. 1, after secure communication channel 154 is established between video conference device 108 and video conference device 128, video conference device 108 is securely receiving audio and video data from video conference device 128, while video conference device 128 is concurrently and securely receiving audio and video data from video conference device 108. However, in accordance with aspects of the present disclosure, video conference device 108 is additionally configured to receive data from detector device 112 and add such data to established communication channel 154. As shown in FIG. 3, detector device 112 is configured to transmit detector data to video conference device 108 via communication channel 106, which may be implemented, for example, as a wireless communication channel. Video conference device 108 may obtain the detector data from detector device 112 by any known method, non-limiting examples of which are disclosed in U.S. provisional patent application No. 63/137,315, filed on Jan. 14, 2021, the entire disclosure of which is incorporated herein by reference. A more detailed discussion of processor 322 of video conference device 108 receiving data from detector device 112 will now be described with reference to FIG. 4B.

FIG. 4B illustrates an exploded view of processor 322 at a time t₁ in one sample embodiment in accordance with aspects of the present disclosure.

As shown in the figure, processor 322 receives an additional packet stream 414, and outputs an additional packet stream 416.

For example, presume that the virtual doctor's visit between user 110 and user 130 has been initiated. Both video conference device 108 and 128 are receiving video and audio data from user 110 and 130, respectively. Additionally, user 130 would also like to see the biological parameters of user 110. After establishing a connection with video conference device 108, as shown in FIG. 3, detector device 112 will then execute detector device program 354 stored on memory 340 in order to transmit the detector data representing the detected biological parameters of user 110 to processor 322 of video conference device 108. The detector data is shown as packet stream 414 in FIG. 4B. Therefore, in accordance with aspects of the present disclosure, processor 322 is now not only being supplied with video and audio data from video conference device 108, as represented by packet stream 406, but is now additionally being supplied with detector data representing the detected biological parameters of user 110 from detector device 112, as represented by packet stream 414.

Video conference device 108 can transmit video/audio data and detector data to video conference device 128 in separate packet streams using any known manner with any known system, a non-limiting example of which may include Web Real-Time Communication (WebRTC). WebRTC is an open-source project that can transmit third party data, such as biological parameter data, through a second communication channel in addition to the teleconference data. For example, using WebRTC, video conference device 108 may be configured to transmit the additional detector data representing the biological parameter data of user 110 to video conference device 128. Video conference device 128 will then provide the audio and video data generated by video conference device 108 in addition to the detector data provided by detector device 112 to user 130.

In some embodiments, detector device 112 is configured to encrypt the detector data by any known encryption method. In such cases, encryptor/decryptor 402 of processor 322 of video conference device 108 is configured to decrypt the detector data received from detector device 112.

Returning to FIG. 2, after detector data is received (S208), the data is encoded (S210). For example, as illustrated in FIG. 3, video data generated by camera 326 and audio data generated by microphone 324 of video conference device 108 are provided to processor 322. Further, detector device 112 provides detector data to video conference device 108. In an example distinct stream embodiment, video conference device 108 transmits the video conference data and the detector data to video conference device 108 via two distinct streams. This will be described in greater detail with reference to FIG. 4B.

As shown in FIG. 4B, the video and audio data of video conference device 108 are illustrated as packet stream 406. Additionally, the detector data of detector device 112 is provided to processor 322 as packet stream 414.

Encoder/decoder 404 may encode packet stream 406, the video and audio data from video conference device 108, by any known encoding method. Similarly, encoder/decoder 404 may encode packet stream 414, the detector data from detector device 112, by any known encoding method.

Returning to FIG. 2, after the data is encoded (S210), the encoded data is encrypted (S212). For example, as shown in FIG. 4B, encryptor/decryptor 402 encrypts the encoded data of packet stream 406, the encoded video and audio data from video conference device encoder/decoder 404, by any known encrypting method. Similarly, encryptor/decryptor 402 encrypts the encoded data of packet stream 414, the encoded detector data from encoder/decoder 404, by any known method.

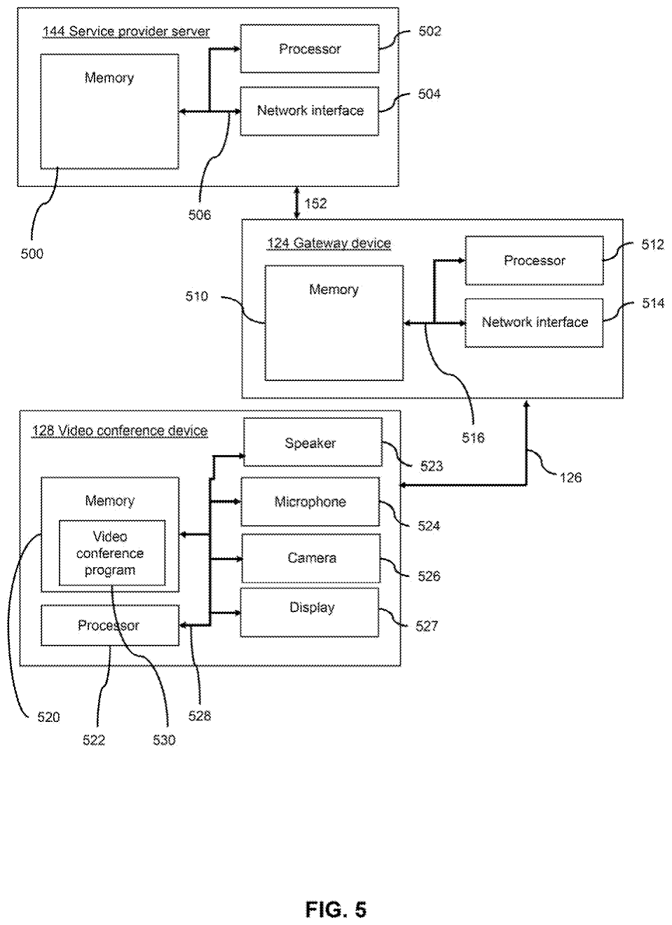

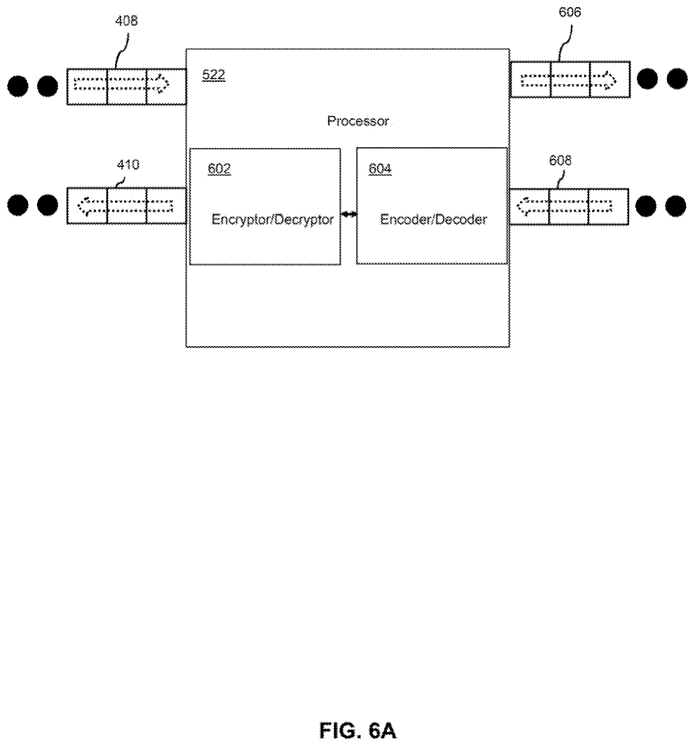

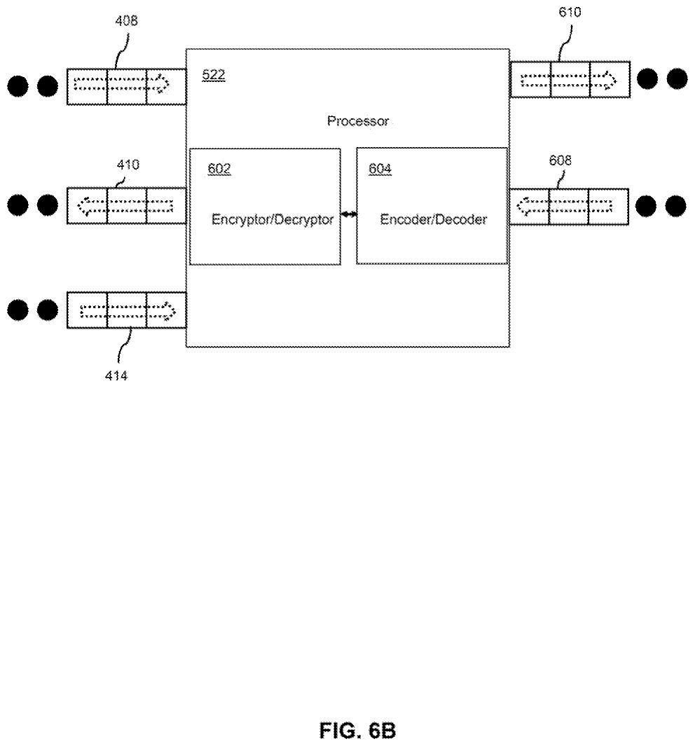

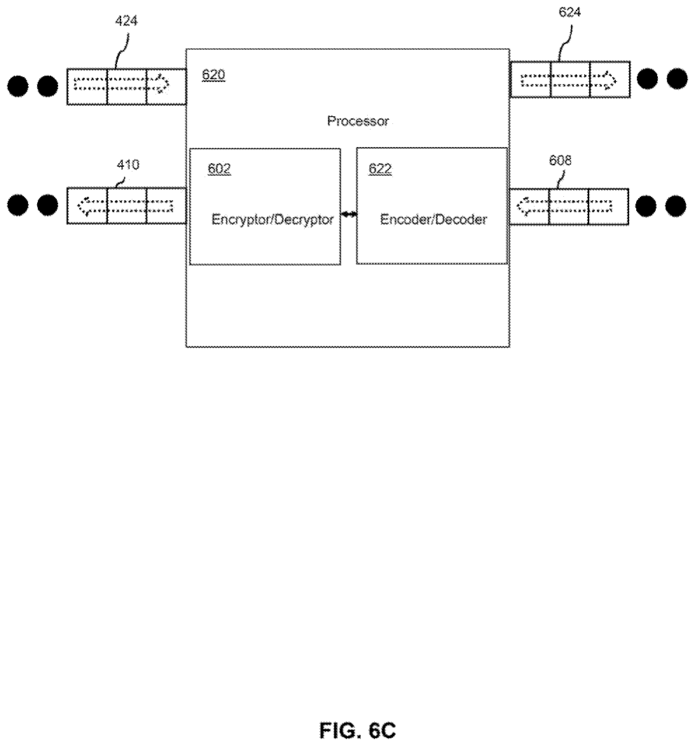

Returning to FIG. 2, after the encoded data is encrypted (S212), the encrypted data is transmitted (S214). For example, as shown in FIG. 4B, processor 322 outputs the encrypted encoded video and audio data of packet stream 406 as packet stream 408. Similarly, processor 322 outputs the encrypted encoded detector data of packet stream 414 as packet stream 416. In this embodiment, processor 322 outputs packet stream 408 and packet stream 416 as distinct streams. In this way the video and audio data generated by video conference device 108 is separate from the detector data generated by detector device 112. Video conference device 108 may then transmit packet stream 408 in a distinct manner from packet stream 416, non-limiting examples of which include via distinct service flows, distinct codecs, distinct communication channels, and combinations thereof. The transmission of packet stream 408 and packet stream 416 will be described in greater detail with reference to FIGS. 5, 6A, and 6B.

FIG. 5 illustrates an exploded view of service provider server 144, gateway device 124, and video conference device 128 in accordance with aspects of the present disclosure.

As shown in the figure, service provider server 144 contains a memory 500, a processor 502, and a network interface 504. Memory 500, processor 502, and network interface 504 are connected by a bus 506. Gateway device 124 contains a memory 510, a processor 512, and a network interface 514. Memory 510, processor 512, and network interface 514 are connected by a bus 516. Video conference device 128 contains a memory 520, a processor 522, a speaker 523, a microphone 524, a camera 526, and a display 527. Memory 520, processor 522, speaker 523, microphone 524, camera 526, and display 527 are connected by bus 528. A video conference program 530 is contained in memory 520 and is executed by processor 522.

For purposes of brevity, service provider server 144 operates in a manner similar to that of service provider 140. Gateway device 124 operates in a manner similar to that of gateway device 104. Video conference device 128 operates in a manner similar to that of video conference device 108, with the exception that video conference device does not receive detector data from a detector within location 122. As there is no detector in location 122, video conference device 128 is not programmed to receive/transmit external data related to an external detector.

FIG. 6A illustrates an exploded view of processor 522 at time t₀ in accordance with aspects of the present disclosure. It should be noted that FIG. 6A illustrates the operation of processor 522 of video conference device 128 in cooperation with the operation of processor 322 of video conference device 108 discussed above with reference to FIG. 4A.

As shown in FIG. 6A, processor 522 includes an encryptor/decryptor 602, an encoder/decoder 604, and packet streams 408, 410, 606, and 608. Encryptor/decryptor 602 is arranged to communicate with encoder/decoder 604.

In this example, encryptor/decryptor 602 and encoder/decoder 604 are illustrated as individual components of processor 522. However, in some embodiments, encryptor/decryptor 602 and encoder/decoder 604 may be combined as a unitary device. Further, in some embodiments, at least one of encryptor/decryptor 602 and encoder/decoder 604 may be implemented as a computer having non-transitory computer-readable media for carrying or having computer-executable instructions or data structures stored thereon. At time t₀, processor 522 is only receiving the audio/video data associated with the secure video conference established with video conference device 108. However, as will be described with reference to FIG. 6B, processor 522 will eventually receive the biometric data additionally from video conference device 108.

FIG. 6B illustrates an exploded view of processor 522 at time t₁ in one sample embodiment in accordance with aspects of the present disclosure. It should be noted that FIG. 6B illustrates the operation of processor 522 of video conference device 128 in cooperation with the operation of processor 322 of video conference device 108 discussed above with reference to FIG. 4B.

As shown in FIG. 6B, processor 522 includes an additional packet stream 414 and packet stream 606 of FIG. 6A has been replaced with packet stream 610.

With reference to FIGS. 1, 4A-B, 5 and 6A-B, video data from camera 526 and audio data from microphone 524 will be transmitted to processor 522 of video conference device 128, shown as packet stream 608. The audio and video data will be encoded by encoder/decoder 604, and then be encrypted by encryptor/decryptor 602. This data will be transmitted to processor 322 of video conference device 108 as packet stream 410, as discussed above with reference to FIG. 6A.

As discussed above with reference to FIG. 6A, processor 522 of video conference device 128 will receive packet stream 408, which contains video and audio data transmitted from video conference device 108. However, with reference to FIG. 6B, at time t₁, processor 522 will additionally receive packet stream 416, containing the biometric parameter data of user 110.

Therefore, in accordance with aspects of the present disclosure, after a secure video conference is established, video conference device 108 may easily obtain detector data from a detector within location 102 and include the detector data in the established secure video conference. In this manner, the additional detector data may be easily viewed by user 130, while maintaining the security of the data by way of the established secure video conference.

Returning to FIG. 2, after the encrypted data is transmitted (S214), other encrypted data is received (S216). In particular, not only is video conference device 108 sending secure information to video conference device 128, but video conference device 108 is concurrently receiving secure information from video conference device 128. For example, as shown in FIG. 4A, processor 322 of video conference device 108 will receive packet stream 410. Packet stream 410 contains video and audio data from video conference device 128.

Returning to FIG. 2, after the encrypted data is received (S216), the encrypted data is decrypted (S218). For example, with reference to FIGS. 4A and 4B, encryptor/decryptor 402 will decrypt packet stream 410 by any known decryption method.

Returning to FIG. 2, after the encrypted data is decrypted (S218), the newly decrypted data is decoded (S220). For example, with reference to FIG. 4B, encryptor/decryptor 402 will decode the decrypted data. This data is then transmitted from processor 322 as packet stream 412, wherein the audio data is transmitted to speaker 323, and the video data is transmitted to display 327.

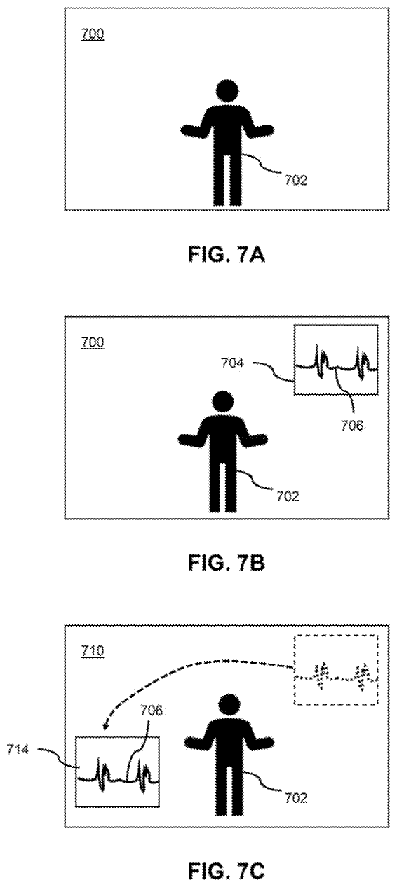

Returning to FIG. 2, after the decrypted data is decoded (S220), the video conference data is provided (S222). This will be described in greater detail with reference to FIGS. 7A-7B.

FIGS. 7A and 7B illustrate sample embodiments of a secure video conference 700 on video conference device 128 in accordance with aspects of the present disclosure. FIG. 7C illustrates a sample embodiment of a secure video conference 710 on video conference device 128 in accordance with aspects of the present disclosure.

As shown in FIG. 7A, secure video conference 700 displays an image 702 of user 110. Image 700 of FIG. 7A corresponds to time t₀ as discussed with reference to FIGS. 4A and 6A above, wherein the video conference only includes audio and video data generated by video conference device 108.

For example, with reference to FIGS. 1, 5, and 7A, while secure video conference 700 is active, video conference device 128 will have a picture of user 110 displayed on display 527 shown as image 702. Video conference device 128 will also have audio emitted from speaker 523. Similarly, video conference device 108 will have a picture of user 130 displayed on display 327, and will have audio emitted from speaker 323.

As shown in FIG. 7B, secure video conference 700 displays image 702 as well as a detector data window 704, and detector data 706. Image 700 of FIG. 7B corresponds to time t₁ as discussed with reference to FIGS. 4B and 6B above, wherein the video conference includes audio and video data generated by video conference device 108 and detector data generated by detector device 112.

However, with reference to FIG. 7B, only video conference device 128 will have detector data window 704 and detector data 706. User 110 uses detector device 112 and video conference device 108 to transmit detector data to video conference device 128. User 130 has no biometric parameter data to transmit, so video conference device 108 will only provide video and audio data of user 130 to user 110.

For example, with reference to FIG. 7B, secure video conference 700 will display detector data 706 in detector data window 704 separately from image 702. In some embodiments, processor 522 of video conference device 128 may not separate the audio data and video data as generated from video conference device 108 from the detector data as provided by detector device 112. For example, as shown in FIG. 6B, in some embodiments, processor 522 may output packet stream 610 such that the data payloads of packet stream 408 and packet stream 414 are combined, wherein the image data generated by video conference device 108 and image data of detector data as provided by detector device 112 are combined. In such cases, as shown in FIG. 7B, detector data window 704 is static as it is part of image 702 and will remain in a fixed location within image 702 in secure video conference 700.

In other embodiments, processor 522 of video conference device 128 may separate the audio data and video data as generated from video conference device 108 from the detector data as provided by detector device 112. This will be described in greater detail with reference to FIG. 7C.

As shown in FIG. 7C, secure video conference 710 displays image 702 as well as a detector data window 714, and detector data 706. However, in this embodiment, detector data window 714 is movable.

For example, as shown in FIG. 6B, in some embodiments, processor 522 may output packet stream 610 such that the data payloads of packet stream 408 and packet stream 414 are separable, wherein the image data generated by video conference device 108 and the image data of detector data as provided by detector device 112 may be identified by processor 522 and may be separately managed. In such cases, as shown in FIG. 7C, secure video conference 710 will display detector data 706 in detector data window 714 separately from image 702. As shown in the figure, detector data window 714 is not static, wherein user 130 has the ability to move detector data window 714 in secure video conference 710.

Returning to FIG. 2, after the video conference data is provided (S222), algorithm 200 ends (S224).

In the above-discussed embodiment with reference to FIG. 4B, processor 322 outputs the audio and video data generated by video conference device 108 and the detector data as provided by detector device 112 as distinct data streams. However, in some embodiments, as will be discussed with reference to FIGS. 4C and 6C, video conference data and detector data may be packaged together into a single data stream.

FIG. 4C illustrates an exploded view of processor 322 at a time t₂ in one sample embodiment in accordance with aspects of the present disclosure.

As shown in the figure, processor 420 includes an encryptor/decryptor 402, an encoder/decoder 422, and packet streams 406, 410, 412, 414, and 424. Processor 420 of FIG. 4C differs from processor 322 of FIG. 4B in that processor 420 outputs a single output packet stream 424 based on a combination of input packet stream 406 and input packet stream 414.

FIG. 6C illustrates an exploded view of processor 522 at a time t₂ in one sample embodiment in accordance with aspects of the present disclosure.

As shown in the figure, processor 620 includes an encryptor/decryptor 602, an encoder/decoder 622, and packet streams 410, 424, 608, and 624. Processor 620 of FIG. 6C differs from processor 522 of FIG. 6B in that processor 620 is able to process the single output packet stream 424 discussed above with reference to FIG. 4C.

For example, presume that the virtual doctor visit between user 110 and user 130 has been initiated. Both video conference device 108 and 128 are receiving video and audio data from user 110 and 130 respectively. However, user 130 would also like to see the biological parameters of user 110. With reference to FIG. 4C, detector device 112 will then execute detector device program 354 stored on memory 340 in order to transmit the biological parameters of user 110 to processor 420 of video conference device 108, shown as packet stream 414. Now, processor 420 is being supplied video and audio data from video conference device 108, in addition to the biometric parameters of user 110 from detector device 112, shown as packet streams 406 and 414 respectively.

Processor 420 will have encryptor/decryptor 402 encrypt packet streams 406 and 414. However, encoder/decoder 422 will encode the encrypted packet streams together, rather than encoding both separately. Once this data is encrypted and encoded, it will be sent out as a single packet stream, shown as packet stream 424.

With reference to FIG. 6C, packet stream 424 is transmitted to processor 620 where it is decrypted by encryptor/decryptor 602, and decoded by encoder/decoder 622. This data is processed and transmitted as packet stream 624, which includes both the video conference data as well as the detector data. This single data stream includes audio data that is distributed to speaker 523, video data that is distributed to display 527, and detector data which is also distributed to display 527.

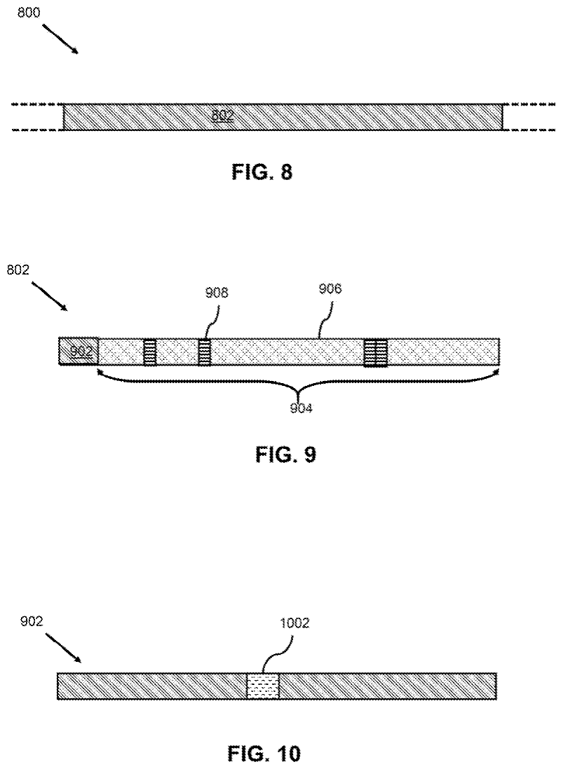

Processor 420 may output packet stream 424 such that image data of detector data as provided by detector device 112 may be identified and may be separately managed by any known method, a non-limiting example of which will be discussed in greater detail with reference to FIGS. 8, 9, and 10.

FIG. 8 illustrates a packet stream 800 in accordance with aspects of the present disclosure.

As shown in the figure, packet stream 800 includes a plurality of packets, an example of which is indicated as packet 802. Packet stream 800 is representative of a combined packet stream that includes the audio and video data generated by video conference device 108 and image data of detector data as provided by detector device 112, such as, for example, packet stream 424 of FIG. 4C and packet stream 624 of FIG. 6B.

FIG. 9 illustrates an exploded view of packet 802 in accordance with aspects of the present disclosure.

As shown in the figure, packet 802 includes a packet header 902 and a payload 904. Payload 904 includes a plurality of sections corresponding to the audio and video data generated by video conference device 108, a sample of which is indicated as video conference data section 906, and a plurality of sections corresponding to the detector data provided by detector device 112, a sample of which is indicated as detector data section 908.

FIG. 10 illustrates an exploded view of packet header 902 in accordance with aspects of the present disclosure.

As shown in the figure, packet header 902 includes a packet identifier (PID) 1002.

PID 1002 identifies the locations of each of detector data sections within in payload 904. By using PID 1002, processor 522 may be able to identify and separate the detector data as provided by detector device 112 from the audio and video data as provided by video conference device 108.

For example, with reference to FIGS. 7C, 8, 9, and 10, video conference device 128 will receive a packet stream 800, which contains many packets, such as packet 802. Packet 802 contains a payload 904, which consists of both the video conference data 906 and detector data 908. Video conference data 906 is audio and video data obtained from microphone 324 and camera 326 of video conference device 108. Detector data 908 contains the detector data obtained from detector device 112. PID 1002 will identify detector data 908 in payload 904, and separate it from video conference data 906, thereby allowing image 702 to be separate from detector data window 714 within secure video conference 710.

In the above discussed non-limiting example embodiments, video conference device 108 obtains detector data from detector device 112 and includes such detector data in an existing secure video conference. However, it should be noted that any number of detectors may be used, wherein video conference device 108 may obtain distinct detector data from a plurality of distinct detectors, respectively, and include such distinct detector data sets in an existing secure video conference.

Due to the pandemic, it has been difficult for people to have in-person doctor appointments, leading to an increase in teleconference doctor appointments. Doctors are not able to physically examine their patients and must rely on the patient to show them their biometric data. This can be a problem, as it may be hard for the patient to show the doctor their biometric data.

In accordance with the present disclosure, a patient will transmit their biometric data from their medical equipment to their video conference device during a secure teleconference. Then, the video and audio data from the video conference device is transmitted simultaneously with the biometric data from the medical equipment to the other video conference device being used by a doctor. The doctor's video conference device will then display the video data from the patient and the biometric data from the medical device.

The present disclosure as disclosed securely transmits biometric data to a doctor over a teleconference.

The foregoing description of various preferred embodiments have been presented for purposes of illustration and description. It is not intended to be exhaustive or to limit the invention to the precise forms disclosed, and obviously many modifications and variations are possible in light of the above teaching. The example embodiments, as described above, were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A first video conference device for use with a detector device, a second video conference device, and a wide area network (“WAN”), the second video conference device being configured to perform a video conference over a secure communication channel over the WAN, to encode second user video data and second user audio data to obtain second encoded data, to encrypt the second encoded data to obtain second encrypted data, to provide the second encrypted data to said first video conference device during the video conference, to receive first encrypted data, to display first user video data based on the first encrypted data, to provide detector data and to play first user audio data based on the first encrypted data, the detector device being configured to provide the detector data to said first video conference device, said first video conference device comprising:
a camera configured to generate the first user video data;
a microphone configured to generate the first user audio data;
a display;
a speaker;
a memory; and
a processor configured to execute instructions stored on said memory to cause said first video conference device to:
receive the first user video data from said camera;
receive the first user audio data from said microphone;
receive the detector data from the detector device;
encode the first user video data, the first user audio data and the detector data to generate the first encoded data;
encrypt the first encoded data to generate first encrypted data;
transmit the first encrypted data to the second video conference device during the video conference;
receive the second encrypted data;
decrypt the second encrypted data to obtain second encoded data;
decode the second encoded data to obtain the second video data and the second audio data;
instruct the display to display second video data based on the second video data; and
instruct the speaker to play second audio data based on the second audio data.

2. The first video conference device of claim 1, wherein said processor is configured to execute instructions stored on said memory to additionally cause said first video conference device to encode the first user video data, the first user audio data and the detector data to generate the first encoded data such that the detector data is encapsulated within at least one of the first user video data and the first user audio data.

3. The first video conference device of claim 1, wherein said processor is configured to execute instructions stored on said memory to additionally cause said first video conference device to:
encode the first user video data and the first user audio data to generate encoded first user video data and encoded first user audio data;
encode the detector data to generate encoded detector data;
encrypt the first user video data and the first user audio data to generate encrypted first user data;
encrypt the encoded detector data to generate encrypted detector data;
transmit the encrypted first user data to the second video conference device via a first communication protocol; and
transmit the encrypted detector data to the second video conference device via a second communication protocol.

4. The first video conference device of claim 1, for use with the detector device being configured to detect a biological parameter and provide a detected signal based on the detected biological parameter.

5. The first video conference device of claim 4, for use with the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

6. A method of using a first video conference device with a detector device, a second video conference device, and a wide area network (“WAN”), the second video conference device being configured to perform a video conference over a secure communication channel over the WAN, to encode second user video data and second user audio data to obtain second encoded data, to encrypt the second encoded data to obtain second encrypted data, to provide the second encrypted data to the first video conference device during the video conference, to receive first encrypted data, to display first user video data based on the first encrypted data, to provide detector data and to play first user audio data based on the first encrypted data, the detector device being configured to provide the detector data to the first video conference device, said method comprising:
receiving, from a camera and via a processor configured to execute instructions stored on a memory, first user video data;
receiving, from a microphone and via the processor, first user audio data;
receiving, from the detector device and via the processor, the detector data;
encoding, via the processor, the first user video data, the first user audio data and the detector data to generate the first encoded data;
encrypting, via the processor, the first encoded data to generate first encrypted data;
transmitting, via the processor, the first encrypted data to the second video conference device during the video conference;
receiving, via the processor, the second encrypted data;
decrypting, via the processor, the second encrypted data to obtain second encoded data;
decoding, via the processor, the second encoded data to obtain the second video data and the second audio data;
instructing, via the processor, a display to display second video data based on the second video data; and
instructing, via the processor, a speaker to play second audio data based on the second audio data.

7. The method of claim 6, wherein said encoding, via the processor, the first user video data, the first user audio data and the detector data to generate the first encoded data comprises encoding the first user video data, the first user audio data and the detector data to generate the first encoded data such that the detector data is encapsulated within at least one of the first user video data and the first user audio data.

8. The method of claim 6, further comprising:
encrypting, via the processor, the first user video data and the first user audio data to generate encrypted user data;
encoding, via the processor, the first user video data and the first user audio data to encoded first user video data and encoded first user audio data;
encoding, via the processor, the detector data to generate encoded detector data;
encrypting, via the processor, the first user video data and the first user audio data to generate encrypted first user data;
encrypting, via the processor, the encoded detector data to generate encrypted detector data;
transmitting, via the processor, the encrypted first user data to the second video conference device via a first communication protocol; and
transmitting, via the processor, the encrypted detector data to the second video conference device via a second communication protocol.

9. The method of claim 6, wherein said receiving the detector data comprises receiving detector data from the detector device being configured to detect a biological parameter and provide a detected signal based on the detected biological parameter.

10. The method of claim 9, wherein said receiving the detector data comprises receiving detector data from the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.

11. A non-transitory, computer-readable media having computer-readable instructions stored thereon, the computer-readable instructions being capable of being read by a first video conference device for use with a detector device, a second video conference device, and a wide area network (“WAN”), the second video conference device being configured to perform a video conference over a secure communication channel over the WAN, to encode second user video data and second user audio data to obtain second encoded data, to encrypt the second encoded data to obtain second encrypted data, to provide the second encrypted data to the first video conference device during the video conference, to receive first encrypted data, to display first user video data based on the first encrypted data, to provide detector data and to play first user audio data based on the first encrypted data, the detector device being configured to provide the detector data to the first video conference device, wherein the computer-readable instructions are capable of instructing the first video conference device to perform the method comprising:
receiving, from a camera and via a processor configured to execute instructions stored on a memory, first user video data;
receiving, from a microphone and via the processor, first user audio data;
receiving, from the detector device and via the processor, the detector data;
encoding, via the processor, the first user video data, the first user audio data and the detector data to generate the first encoded data;
encrypting, via the processor, the first encoded data to generate first encrypted data;
transmitting, via the processor, the first encrypted data to the second video conference device during the video conference;
receiving, via the processor, the second encrypted data;
decrypting, via the processor, the second encrypted data to obtain second encoded data;
decoding, via the processor, the second encoded data to obtain the second video data and the second audio data;
instructing, via the processor, a display to display second video data based on the second video data; and
instructing, via the processor, a speaker to play second audio data based on the second audio data.

12. The non-transitory, computer-readable media of claim 11, wherein the computer-readable instructions are capable of instructing the first video conference device to perform the method wherein said encoding, via the processor, the first user video data, the first user audio data and the detector data to generate the first encoded data comprises encoding the first user video data, the first user audio data and the detector data to generate the first encoded data such that the detector data is encapsulated within at least one of the first user video data and the first user audio data.

13. The non-transitory, computer-readable media of claim 11, wherein the computer-readable instructions are capable of instructing the first video conference device to perform the method further comprising:
encrypting, via the processor, the first user video data and the first user audio data to generate encrypted user data;
encrypting, via the processor, the detector data to generate encrypted detector data;
encoding, via the processor, the encrypted user data to generate the encrypted encoded user video data and encrypted encoded user audio data;
encoding, via the processor, the encrypted detector data to generate the encrypted encoded detector data;
transmitting, via the processor, the encrypted encoded user video data and encrypted encoded user audio data to the second video conference device via a first communication protocol; and
transmitting, via the processor, the encrypted encoded detector data to the second video conference device via a second communication protocol.

14. The non-transitory, computer-readable media of claim 11, wherein the computer-readable instructions are capable of instructing the first video conference device to perform the method wherein said receiving the detector data comprises receiving detector data from the detector device being configured to detect a biological parameter and provide a detected signal based on the detected biological parameter.

15. The non-transitory, computer-readable media of claim 11, wherein the computer-readable instructions are capable of instructing the first video conference device to perform the method wherein said receiving the detector data comprises receiving detector data from the detector device being selected from the group of detector devices consisting of cameras, microphones, pressure sensors, blood-pressure sensors, chemical detectors, oxygen sensors, carbon dioxide sensors, heart sound sensors, blood-flow sensors, respiration sensor, electrochemical electrodes, electrocardiograms, and combinations thereof.
